(19) United States
(12) Patent Application Publication
(10) Pub. No.: US 2011/0288260 A1
(43) Pub. Date: Nov. 24, 2011

(54) MAIN-CHAIN BENZOXAZINE OLIGOMER COMPOSITIONS, AND METHOD FOR THE PREPARATION THEREOF

(76) Inventors: Hatsuo Ishida, Cleveland, OH (US); Tarek Agag, Cleveland, OH (US)

(21) Appl. No.: 13/050,639

(22) Filed: Mar. 17, 2011

Related U.S. Application Data
(60) Provisional application No. 61/315,709, filed on Mar. 19, 2010.

(51) Int. Cl. C08G 14/073; C07D 413/14

(52) U.S. Cl. 528/153; 544/90

(57) ABSTRACT

Disclosed are benzoxazine compositions where oxazine rings are part of the polymer main-chain. The benzoxazine oligomers are prepared by the reaction of a bisphenol and an aldehyde, such as formaldehyde or paraformaldehyde, with one or more diamines or polyamines, and optionally with one or more monofunctional amines or monofunctional phenols. Also disclosed are benzoxazine monomers prepared from a bisphenol, an aldehyde, such as formaldehyde or paraformaldehyde, and a monofunctional amine, which benzoxazine monomers may be optionally used as reactive diluents. Further disclosed are benzoxazine monomers are prepared from a diamine, and aldehyde, such as formaldehyde or paraformaldehyde, and a monofunctional phenol, which benzoxazine monomers may also be optionally used as reactive diluents.

### RELATED APPLICATION DATA

This application claims benefit to U.S. Provisional Application No. 61/315,709, filed Mar. 19, 2010, of which the entire contents of the application are incorporated by reference herein.

### FIELD OF THE INVENTION

The invention relates to main-chain benzoxazine compositions, and to their preparation from a bisphenol, an aldehyde, and a diamine or polyamine.

### BACKGROUND OF THE ART

Polybenzoxazines (PBZs) have attracted attention because of excellent mechanical and thermal properties, with good handling capability for material processing and composite manufacturing. The polymerization of benzoxazines can be achieved through the cationic ring opening of the oxazine ring, with or without an added initiator or catalyst. Another unique characteristic is that polybenzoxazines have greater molecular design flexibility than other polymers. They release no reaction by-product during polymerization reactions, and no strong acid or alkaline catalysts are required for the synthesis of monomers or polymerization. However, some acids, such as phenols and carboxylic acids, as is known in the art, will accelerate the rate of polymerization. Furthermore, no volatiles are released and almost no shrinkage is achieved upon polymerization.

However, the synthesis of main-chain benzoxazine polymers through Mannich base polycondensation is associated with shortcomings, such as insolubility of the products due to molecular rigidity, resulting in a low molecular weight and broad polydispersity. There is a need in the art for main-chain benzoxazine oligomers, having excellent mechanical and physical properties, as well as ease of processing. There is also a need in the art for lower cost main-chain benzoxazine oligomers.

### SUMMARY OF THE INVENTION

In one aspect, the invention relates to main-chain benzoxazine oligomers prepared by the reaction of a bisphenol, an aldehyde, one or more diamines or polyamines, and optionally one or more monofunctional amines or monofunctional phenols.

In another aspect, the invention relates to benzoxazine monomers prepared by the reaction of a bisphenol, an aldehyde, and a monofunctional amine, which benzoxazine monomers may be optionally used as reactive diluents.

In another aspect, the invention relates to benzoxazine monomers prepared from a diamine, an aldehyde, and a monofunctional phenol, which benzoxazine monomers may also be optionally used as reactive diluents.

### SUMMARY OF THE FIGURES

FIG. 1 shows ¹H NMR spectra of benzoxazine monomers and main-chain oligomers.

FIG. 2a shows ¹³C NMR spectra of benzoxazine monomers.

FIG. 2b shows ¹³C NMR spectra of main-chain benzoxazine oligomers.

FIG. 3 shows IR spectra of benzoxazine monomers and main-chain oligomers.

FIG. 4 is a graph showing viscosity (Pa·s) of benzoxazine resins as a function of Temperature (° C.).

FIG. 5 shows DSC thermograms of a benzoxazine monomer and a main-chain oligomer.

FIG. 6 is a graph showing storage modulus (MPa) of a benzoxazine monomer and a main-chain oligomer as a function of temperature (° C.).

FIG. 7 is a graph showing loss moduli (MPa) of a benzoxazine monomer and a main-chain oligomer as a function of temperature (° C.).

FIG. 8 is a graph showing Tan δ of the benzoxazine monomer and main-chain oligomer as a function of temperature (° C.).

FIG. 9 shows TGA thermograms of the benzoxazine monomer and main-chain oligomer.

### DESCRIPTION OF THE PREFERRED EMBODIMENTS

A new class of benzoxazines, where oxazine rings are part of the polymer main-chain, has been developed. In one embodiment of the application, benzoxazine oligomers are prepared by the reaction of a bisphenol and an aldehyde, such as formaldehyde or paraformaldehyde, with one or more diamines or polyamines, and optionally with one or more monofunctional amines or monofunctional phenols. In another embodiment of the application, benzoxazine monomers are prepared from a bisphenol, and aldehyde, such as formaldehyde or paraformaldehyde, and a monofunctional amine, which benzoxazine monomers may be optionally used as reactive diluents. In another embodiment of the application, benzoxazine monomers are prepared from a diamine, and aldehyde, such as formaldehyde or paraformaldehyde, and a monofunctional phenol, which benzoxazine monomers may also be optionally used as reactive diluents.

In one embodiment, the bisphenol is a compound containing two hydroxyphenyl functionalities. Examples of suitable bisphenols include but are not limited to bisphenol A (2,2-bis(4-hydroxyphenyl)propane), bisphenol AP (1,1-bis(4-hydroxyphenyl)-1-phenyl-ethane), bisphenol AF (2,2-bis(4-hydroxyphenyl)hexafluoropropane), bisphenol B (2,2-bis(4-hydroxyphenyl)butane), bisphenol BP (bis-(4-hydroxyphenyl)diphenylmethane), bisphenol C (bis(4-hydroxyphenyl)-2,2-dichloroethylene and also 2,2-bis(3-methyl-4-hydroxyphenyl)propane), bisphenol E (1,1-bis(4-hydroxyphenyl)ethane), bisphenol F (bis(4-hydroxyphenyl)methane), bisphenol G (2,2-bis(4-hydroxy-3-isopropylphenyl)propane), bisphenol M (4,4′-(1,3-phenylenediisopropylidene) bisphenol), bisphenol S (bis(4-hydroxyphenyl)sulfone), bisphenol P (4,4′-(1,4-phenylenediisopropylidene) bisphenol), bisphenol TMC (1,1-bis(4-hydroxyphenyl)-3,3,5-trimethylcyclohexane), and bisphenol Z (1,1-bis(4-hydroxyphenyl)-cyclohexane), including any combination or subset of the above.

In one embodiment, the aldehyde may be an aldehyde added directly or formed in situ. In another embodiment, formaldehyde may be used or formed in situ. In another embodiment, paraformaldehyde, polyoxymethylene, hexamethylenetetramine, or trioxane may be used as a compound to form formaldehyde in situ.

In one embodiment, the diamine or polyamine is an alkyl or aromatic diamine, including aliphatic or aromatic ether diamines, which may be substituted or unsubstituted, or any mixture or subset thereof. Examples of suitable diamines or polyamines include but are not limited to 4,4′-oxydianiline, 4,4′-diaminodiphenylmethane, 1,4-bis-(4-aminophenoxy)benzene, 4,4′-(p-biphenylenedioxy)dianiline, 4,4′-(9H-fluorene-9,9-diyl)dianiline, 2,7-bis(4-aminophenoxy)naphthalene, 1,3-bis(3-aminophenoxy)benzene, 1,3-bis(4-aminophenoxy)benzene, 1,4-bis(3-aminophenoxy)benzene, 1,3-bis(4-aminophenoxy)neopentane, 2,2-bis[4-(3-aminophenoxy)phenyl]propane, 2,2-bis[4-(4-aminophenoxy)phenyl]propane, 4,4′-bis(3-aminophenoxy)biphenyl, 4,4′-bis(4-aminophenoxy)biphenyl, 2,2-bis[4-(4-aminophenoxy)phenyl]hexafluoropropane, bis[3-(3-aminophenoxy)phenyl]ether, bis[3-(4-aminophenoxy)phenyl]ether, bis[4-(3-aminophenoxy)phenyl]ether, bis[4-(4-aminophenoxy)phenyl]ether, and combinations or subsets thereof.

In another embodiment, the diamine or polyamine is a linear moiety having at least three unsubstituted amine groups. Examples of these include but are not limited to oligomeric aniline-formaldehyde condensates and their hydrogenated counterparts, and the like, having a molecular weight of from about 200 to about 5000 daltons.

In one embodiment, in the preparation of benzoxazine monomers, the monofunctional amine is a monoprimary aliphatic or aromatic amine containing between about 2 to about 50 carbon atoms, which may optionally contain one or more of ether, thioether, secondary amine, or tertiary amine linkages.

In another embodiment, in the preparation of benzoxazine monomers, the monofunctional phenol is phenol or a substituted or unsubstituted phenolic compound.

In one embodiment, in the preparation of the main-chain benzoxazine oligomer, the bisphenol, diamine or polyamine, and aldehyde are contacted at a temperature of between about 50° C. and about 100° C., for 1 to 10 hours, in a mole ratio of about 0.8:0.8:3.3 to about 1.2:1.2:4.8.

In one embodiment, in the preparation of the benzoxazine monomer, the bisphenol, monofunctional amine, and aldehyde are contacted at a temperature of between about 50° C. and about 100° C., for 1 to 10 hours, in a mole ratio of about 0.8:1.6:3.3 to about 1.2:2.4:4.8.

In one embodiment, in the preparation of the benzoxazine monomer, the diamine, monofunctional phenol and aldehyde are contacted at a temperature of between about 50° C. and about 100° C., for 1 to 10 hours, in a mole ratio of about 0.8:1.6:3.3 to about 1.2:2.4:4.8.

In one embodiment, the main-chain benzoxazine oligomers have a number average molecular weight (Mₐ) of between about 300 and about 10,000. In another embodiment, the main-chain benzoxazine oligomers have a number average molecular weight (Mₙ) of between about 300 and about 7,000. It is well known to those skilled in the art to control molecular weight by manipulation of the reaction stoichiometry.

In another embodiment, it may be desirable for the main-chain benzoxazine oligomers of the invention to contain a non-reactive end-group. In one such embodiment the benzoxazine oligomers are prepared by the reaction of a bisphenol, an aldehyde, one or more diamines or polyamines and a monofunctional compound, which monofunctional compound may be, for example, either a monofunctional amine or a monofunctional phenol, as described above.

In one embodiment, the main-chain benzoxazine oligomers have a degree of polymerization (DP) less than about 15. In another embodiment, the DP is between about 1 and about 15. In another embodiment, the DP is between about 1 and about 10.

In one embodiment, the main-chain benzoxazine oligomers have a polydispersity index (PDI) of between about 1 and about 3, where the PDI is the ratio of the weight average molecular weight (M_{w}) and the number average molecular weight (Mₙ). In another embodiment, the PDI is about 2.

For illustrative purposes, the main-chain benzoxazine compositions of the invention may be prepared according to reaction schemes 1a and 1b below.

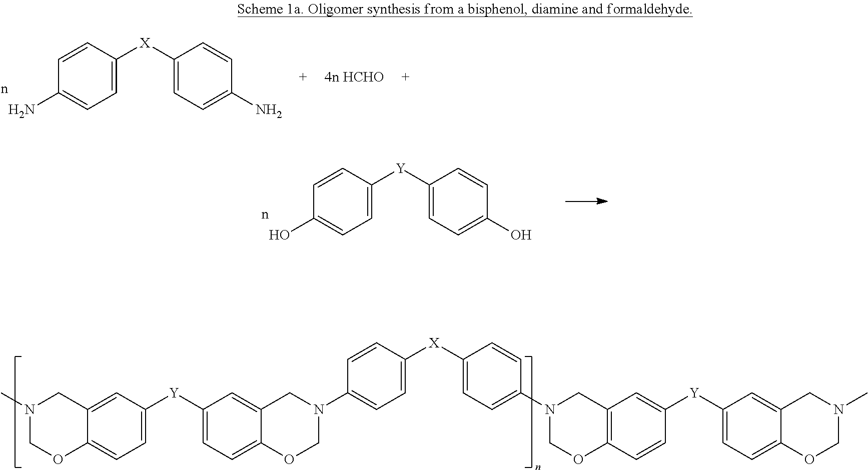

In Reaction Scheme 1a, n is from 1 to 15, X is oxygen, an alkyl, alkylene, alkylidene, or arylene group, an alkylenedioxy group, a heteroatom containing group, or a combination or subset thereof, and Y is an alkyl, alkylene, alkylidene, or aryl group, which may be substituted or halogenated, a heteroatom containing group, or a combination or subset thereof.

In one embodiment X is selected from one or more of the following groups:

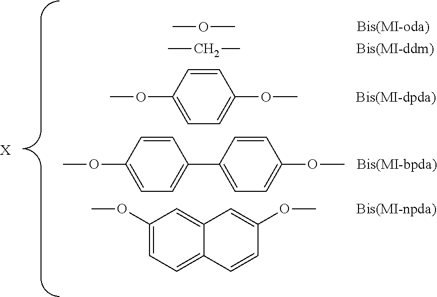

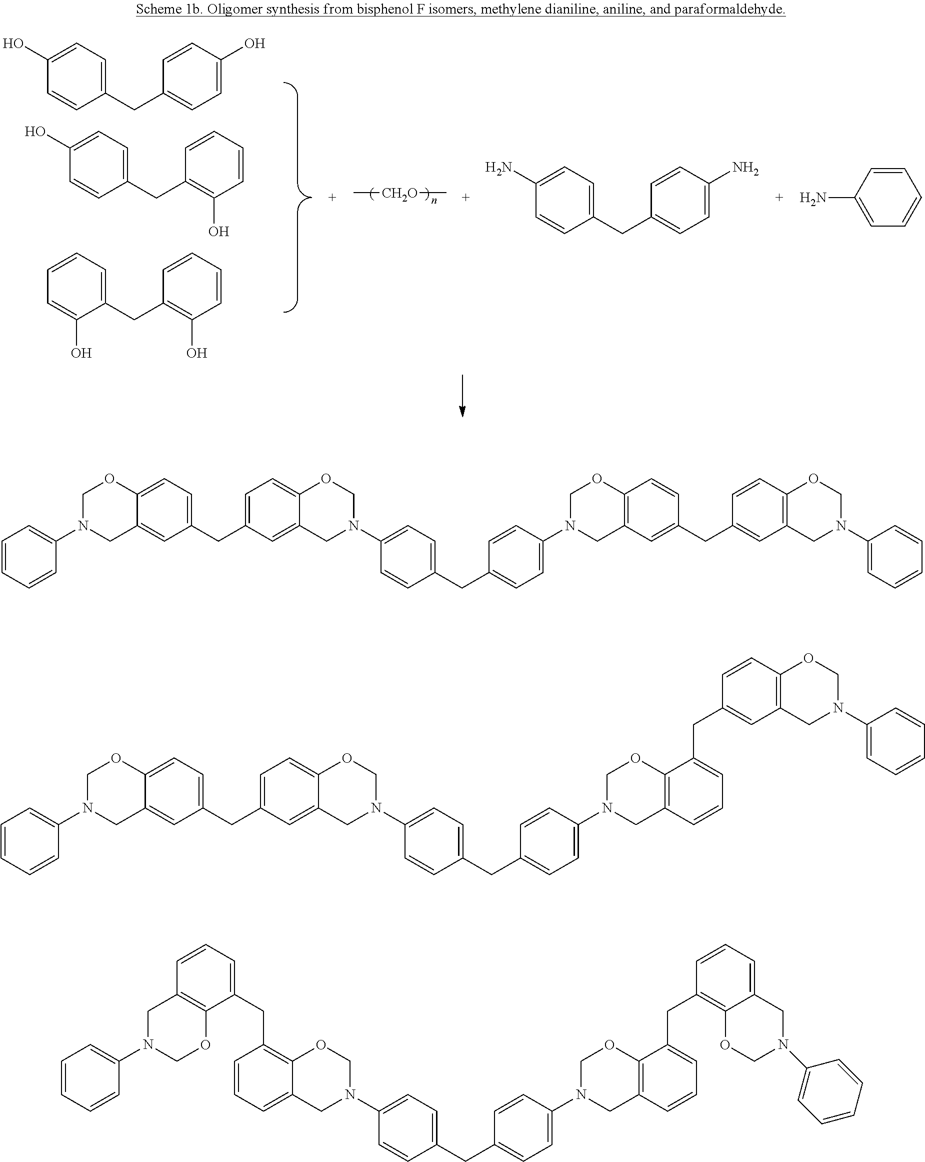

Also for illustrative purposes, benzoxazine monomers, which may be optionally used as reactive diluents, may be prepared according to reaction scheme 2.

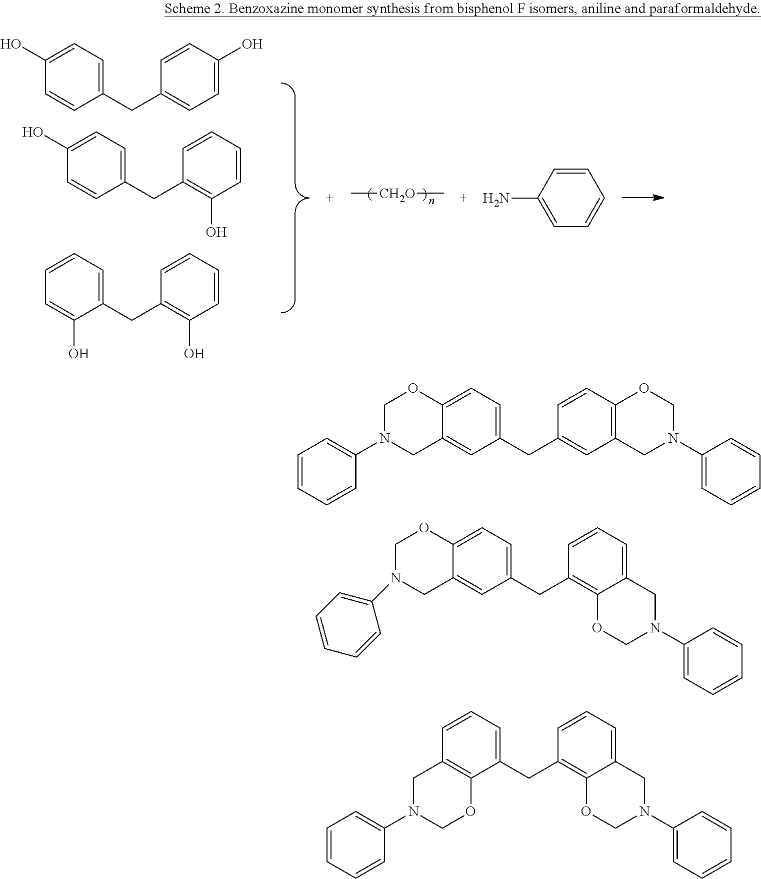

The main-chain benzoxazine oligomers may be polymerized via thermal activation, as is known in the art. In one embodiment, the oligomers undergo ring-opening polymerization from the benzoxazine moiety. In another embodiment, the polymerization is conducted at a temperature less then the polymer degradation temperature. In still another embodiment, the polymerization is conducted at a temperature less then about 250° C.

In one embodiment, the compositions of the invention may be included in resin systems used in the manufacture of structural composites, for example, in the automotive, marine and aerospace industries. In another embodiment, the compositions of the invention may be included in resin systems used in the manufacture of electrical laminates, such as used in printed circuit boards. Such resin systems may be advantageously employed in a wide variety of different manufacturing processes for such structural composites including prepreg, filament winding, pultrusion, resin infusion (various techniques including RTM (Resin Transfer Molding), VARTM (Vacuum Assisted Resin Transfer Molding), SCRIMP (Seeman Composites Resin Infusion Molding Process) etc.), compression molding and hand lay-up.

### EXAMPLES

The following examples are provided to illustrate the present invention. The examples are not intended to limit the scope of the present invention and they should not be so interpreted. Amounts are in weight parts or weight percentages unless otherwise indicated.

### Materials

Mixed isomers of bisphenol-F are produced by Momentive Specialty Chemicals Inc. Aniline (99%), paraformaldehyde (96%) and 4,4′-diaminodiphenylmethane (DDM) (>99%) are commercially available from Aldrich Chemical Company. N,N′-dimethylformamide (DMF), toluene, xylene (o-, m-, p-isomers), hexanes (a mixture of isomers), tetrahydrofuran, methanol and ethanol are commercially available from Fisher Scientific Company.

### Measurements

¹H-NMR spectra were acquired in deuterated dimethylsulfoxide with tetramethylsilane as an internal standard on a Varian Oxford AS300 at a proton frequency of 300 MHz. The average number of transients for ¹H-NMR is 64. A relaxation time of 10 seconds was used for the integrated intensity determination of ¹H-NMR spectra.

TA Instruments DSC model 2920 was used with a heating rate of 10° C./min and a nitrogen flow rate of 60 mL/min for all differential scanning calorimetric (DSC) tests. All samples were crimped in hermetically sealed aluminum pans with lids. Dynamic mechanical analyses (DMA) were done on a TA Instruments Q800 dynamic mechanical analyzer applying controlled strain tension mode with an amplitude of 10 μm and a ramp rate of 3° C./min. Thermogravimetric analyses (TGA) were performed on a TA Instruments Q500 TGA with a heating rate of 10° C./min in a nitrogen atmosphere at a flow rate of 40 mL/min. Viscosity was measured using Anton Paar MCR501 Rheometer with a constant shear rate of 100 s⁻¹ at different elevated temperatures. The sample was spread in a plate to plate holder with a diameter of 50 mm. Polymer molecular weight was determined by size exclusion chromatography (SEC), also known as gel permeation chromatography (GPC), using a Waters GPC system equipped with an LDC/Milton Roy max N series UV detector. The measurements were taken at 25° C. with THF as a mobile phase on two Phenomenex Phenogel columns (100 and 10 nm). Molecular weight was calculated using a calibration based on monodisperse polystyrene standards.

### Example 1

### Preparation of a Bisphenol-F Benzoxazine Monomer Mixture

In a 250 ml round bottom flask were dissolved bisphenol-F isomers (80 mmol, 16.02 g), and aniline (160 mmol, 14.90 g) in 75 mL toluene. Paraformaldehyde (320 mmol, 9.60 g) was added to the solution followed by heating the mixture at 90° C. in a preheated oil bath. By following the reaction by ¹H NMR, it was found that the best conversion to cyclic benzoxazine structure was achieved after about 5 hours. The solution was cooled and precipitated in hexanes to obtain a yellow powder. The powder was finally placed in a vacuum oven at 40° C. for 72 h to dry (Yield: 32 g, 81%).

### Example 2

### Preparation of a Main-Chain Benzoxazine Oligomer Mixture

A mixture of bisphenol-F isomers (100 mmol, 20.02 g), aniline (100 mmol, 9.13 g) DDM (50 mmol 9.90 g) and paraformaldehyde (400 mmol, 12 g) was added in a 250 mL round bottom flask with 100 mL toluene as solvent. The milky mixture was heated gradually and was kept stirring at 90° C. for 5 hours. The formation of triaza gel was observed after about 15 minutes. After 3 hours, the insoluble white color gel disappeared to form a transparent yellow solution. The reaction mixture was stirred for another 3 hours under the same conditions. Then it was cooled to room temperature and then poured into hexanes to obtain a yellow powder. The powder was redissolved in tetrahydrofuran and reprecipitated in methanol to yield a pale yellow powder (Yield: 36 g, 71%).

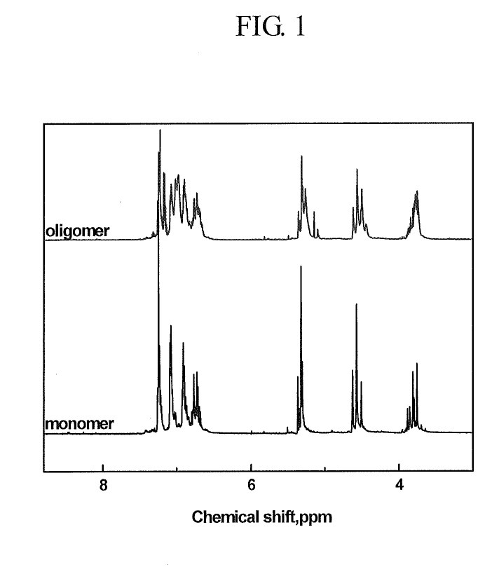

FIG. 1 shows the ¹H-NMR spectra of benzoxazine monomers and main-chain oligomers prepared in Examples 1 and 2. Typically, benzoxazine monomers have two equal intensity singlet peaks in ¹H-NMR spectra due to the CH₂ groups in the oxazine ring. These peaks, however, become broader in main-chain benzoxazine polymers due to the reduced molecular mobility. In the case of the current bisphenol-F study, each characteristic CH₂ resonance of the oxazine ring appears as two equivalent multiplets due to the isomeric nature of bisphenol-F as the starting material for benzoxazine synthesis. Two multiplets are observed at 4.52 and 4.56 ppm for the monomer mixture and 5.30 and 5.34 ppm for the oligomer mixture, which are assigned to (aromatic)-CH₂—N— and —O—CH₂—N—, respectively. The multiplet in the range of 6.58-7.48 ppm is assigned to the protons of the aromatic ring. The multiplet near 3.8 ppm in the monomers and 3.7 ppm in the oligomers corresponds to the CH₂ group between the benzene rings in the bisphenol-F isomers.

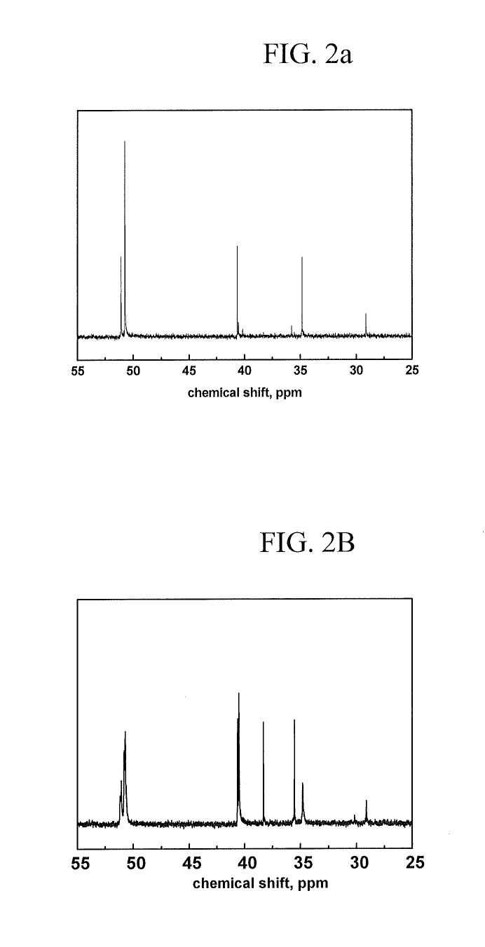

FIGS. 2a and b show the ¹³C-NMR spectra of the monomers and oligomers prepared above. The multiple resonances at 41, 35, and 29 ppm represent the isomer mixture based on p,p′-. o,p′- and o,o′-isomers, respectively.

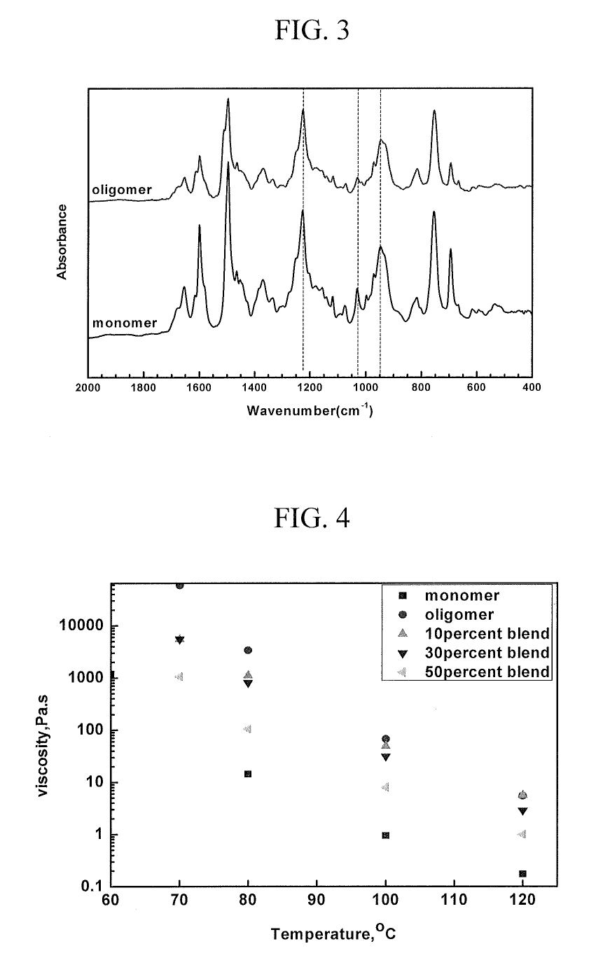

The structures of the benzoxazine monomers and main-chain oligomers prepared above were further confirmed by FTIR. There are a number of infrared bands in the spectra highlighted in FIG. 3, which can be used as characteristic bands of the benzoxazine structure. The presence of a cyclic ether of the benzoxazine structure is confirmed by the absorbance peaks at 1228 and 1035 cm⁻¹ due to the C—O—C asymmetric and symmetric stretching modes, respectively. The characteristic mode of a benzene ring with a fused oxazine ring is located at 949 cm⁻¹. The two peaks around 750 cm⁻¹ and 690 cm⁻¹ indicate a monosubstituted benzene ring, which corresponds to the structure of an oxazine ring attached to aniline. Two bands at 1650 and 1600 cm⁻¹ correspond to the in-plane carbon-carbon stretching of the trisubstituted benzene ring.

The molecular weight of the oligomers prepared in Example 2 was evaluated using SEC and the results are summarized in Table 1. The number average molecular weight (Mₙ) of the oligomer was estimated to be 3208, with a polydispersity index (PDI) equal to 1.99. The degree of polymerization (DP) was calculated based on the proposed structures shown in Scheme 1. This indicates that the synthesized oligomer is a series of short chain mixtures with low molecular weight.

TABLE 1
SEC data for synthesized oligomers

| | Mₙ (Daltons) | M_{w} (Daltons) | PDI | DP |
|---|---|---|---|---|
| Oligomer | 3208 | 6395 | 1.99 | 3 |

### Example 3

### Preparation of Benzoxazine Blend for Viscosity Measurements

Homogeneous blends of monomers and oligomers were made at the monomer-to-oligomer ratios of 10%, 30% and 50% in chloroform and then placed in a vacuum oven for 72 hours at 40° C. to remove the solvent. The dried samples were examined by ¹H-NMR to evaluate the dryness of the solvent and only the ones that showed negligible solvent contents were used for the viscosity tests.

The dynamic viscosities for the benzoxazines, including the neat oligomers, monomers, and oligomer/monomer blend are shown in FIG. 4. A low viscosity benzoxazine monomer can be used as a reactive diluent. In the mixture of benzoxazine monomers and oligomers, both benzoxazines can polymerize through ring-opening reactions yielding a cross-linked product. Furthermore, by mixing benzoxazine monomers with benzoxazine oligomers, one can adjust and control the viscosity of the mixture through varying the ratio of two benzoxazines in the mixture. For the weight ratios of the benzoxazine monomer to benzoxazine oligomer of 1:9, 3:7 and 5:5 the viscosities of the mixture were measured.

Table 2 shows the viscosity change with temperature and the monomer/oligomer mixture ratio. For the benzoxazine monomers, 60° C. was the lowest temperature for the instrument with current set-up to obtain a reliable viscosity value. On the other hand, for the oligomer and reactive diluent mixtures, temperatures above 70° C. were needed to obtain the viscosity value. For the 50% mixture, the viscosity can reach as low as 1 Pa·s at 120° C.

TABLE 2
Viscosity of benzoxazine precursors

| | | | 10% | 30% | 50% |
|---|---|---|---|---|---|
| | | | Monomer | Monomer | Monomer |
| | Monomer/ | Oligomer/ | mixture/ | mixture/ | mixture/ |
| | Pa · s | Pa · s | Pa · s | Pa · s | Pa · s |
| 60 | 1190 | — | — | — | — |
| 70 | — | 58900 | 5525 | 5530 | 1059 |
| 80 | 14.4 | 3390 | 1115 | 804 | 105 |
| 100 | 0.947 | 67.6 | 49.1 | 31.5 | 8 |
| 120 | 0.175 | 5.50 | 5.61 | 2.89 | 1 |

### Example 4

### Polymerization of Benzoxazine Oligomers with and without Reactive Diluents

A solution method was used to prepare samples in the film forms. Solutions of 30% solid content of the monomers in DMF were prepared. Then, the solutions were cast over dichlorodimethylsilane-pretreated glass plates. The films were dried in an air circulating oven at 60° C. for 24 h to remove the solvent. The films as fixed on glass plates were heated stepwise at 110° C., 130° C., 160° C., 200° C. and 220° C. for 2 hours each, and then slowly cooled to room temperature. The films had a brown color with thickness ranging from 0.1 to 0.8 mm.

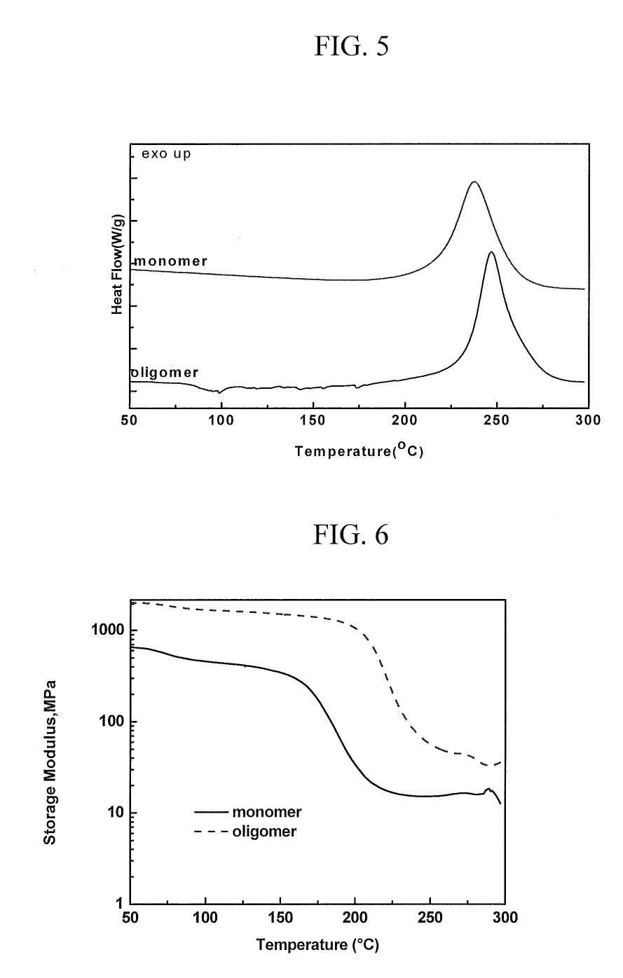

The polymerization behavior of the monomers and oligomers was studied by DSC by stepwise heating at 110° C., 160° C., 200° C., and 220° C. for 2 hrs each. The final polymerization temperature was restricted to 220° C. in order to prevent possible degradation. DSC thermograms are shown in FIG. 5 and the thermal properties are summarized in Table 3. The DSC trace of the monomers shows an exotherm with the onset at 177° C. and a maximum at 238° C. The heat of polymerization is 190 J/g. For the oligomers, one exotherm was observed with the onset at 176° C. and a maximum at 247° C. The heat of polymerization is 238 J/g which maybe due to a higher concentration of the oxazine rings per unit weight in the benzoxazine oligomers.

TABLE 3
Thermal properties of benzoxazine monomers and main-chain oligomers

| | Onset (° C.) | Max (° C.) | Heat of Polymerization (J/g) |
|---|---|---|---|
| Monomer | 177 | 238 | 190 |
| Oligomer | 176 | 247 | 238 |

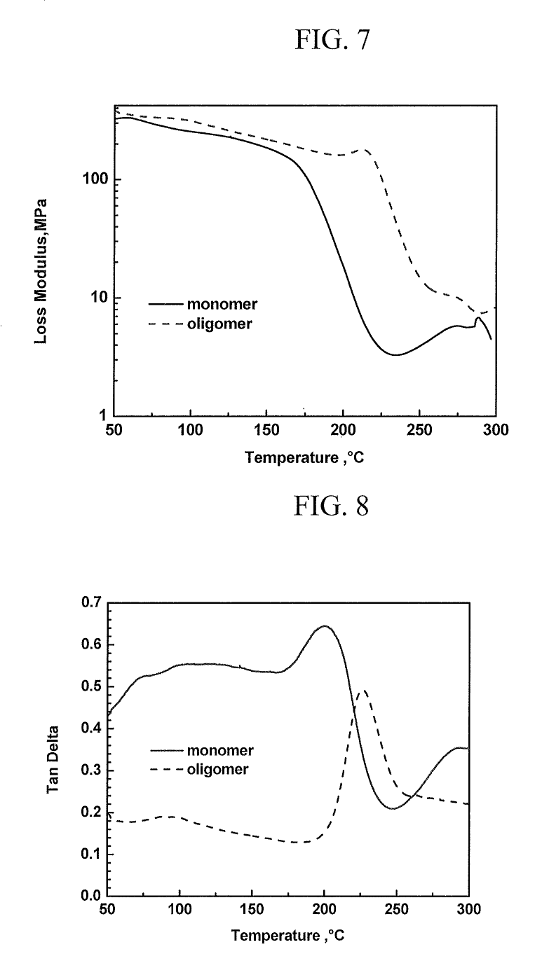

The viscoelastic properties were determined by dynamic mechanical analysis (DMA). FIGS. 6 and 7 show the temperature dependence of the storage moduli and loss moduli for the polybenzoxazine films. For the thermoset polymerized from benzoxazine monomers, the storage modulus is maintained at approximately the same value for a wide temperature range up to 150° C. The glass transition temperature (T_{g}) determined as the peak temperature of the δ-transition of the loss modulus is around 154° C. On the other hand, the polybenzoxazine crosslinked from benzoxazine oligomers has a T_{g} of 213° C. The significant increase in T_{g} by about 60° C. of the crosslinked polymers from oligomers is due to the presence of the difunctional amine linkage, which helps to anchor the amine portion into the network and increases the chain rigidity. Typical glass transition temperature of the fully thermally polymerized bisphenol-A and aniline (BA-a) polybenzoxazine is about 170° C. and char yield at 800° C. under nitrogen environment is about 30%. Tan δ of the benzoxazine monomers and main-chain oligomers is shown in FIG. 8.

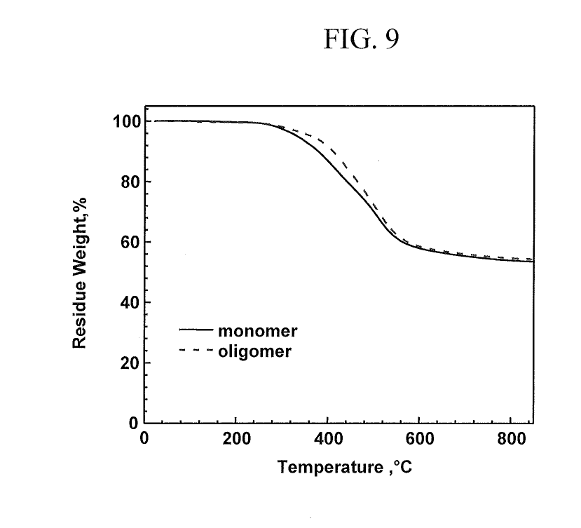

The thermal stability of the novel crosslinked polybenzoxazines is determined by thermogravimetric analysis (TGA). FIG. 9 shows the TGA thermograms of crosslinked polymer films under N₂ atmosphere. The results are summarized in Table 4. For all the polymers studied, the 5% weight loss temperature, T_{d5}, varies from 332° C. to 364° C. The thermal degradation of polybenzoxazines has two main stages: evaporation of the amine followed by the simultaneous breakage of the phenolic linkage and degradation of the Mannich base. Volatilization of aniline from the bisphenol-F/aniline based benzoxazine monomer is relatively easy in comparison to the volatilization of DDM from DDM-based benzoxazine oligomers. Hence, the polybenzoxazines derived from the oligomers show an increase in T_{d5} compared with polybenzoxazines derived from the monomers.

The char yield is defined in this work as the residual weight of the material at 800° C. under N₂. For the crosslinked polybenzoxazines, the char yield for the monomers and oligomers is 53% and 55%, respectively, which is much higher than that of the phenol-DDM-based polybenzoxazine at 46%. The crosslinked polybenzoxazine derived from the oligomers has higher char yield than the reference benzoxazine monomers as the degradation of the monomer at higher temperature (around 500° C.) is more intensive compared with the oligomers. The intermediate temperature stability in the range between 300° C. and 500° C. is significantly higher in the oligomer-derived polybenzoxazine than the monomer-derived.

TABLE 4
Weight loss and char yield of polybenzoxazines

| | 5% weight loss | 10% weight loss | |
|---|---|---|---|
| | temperature (° C.) | temperature (° C.) | Char yield (%) |
| Monomer | 332 | 378 | 52 |
| Oligomers | 346 | 411 | 55 |

We claim:

1. Benzoxazine oligomers comprising a reaction product of a bisphenol, an aldehyde and a diamine or polyamine, wherein the benzoxazine oligomers have a polymeric main-chain containing oxazine rings, and wherein the bisphenol, the diamine or polyamine, and the aldehyde are present in a mole ratio of about 0.8:0.8:3.3 to about 1.2:1.2:4.8.

2. The benzoxazine oligomers of claim 1, wherein the reaction product further comprises a monofunctional amine or a monofunctional phenol in an amount calculated such that the benzoxazine oligomer contains a non-reactive end-group.

3. The benzoxazine oligomers of claim 1, wherein the bisphenol contains a compound selected from the group consisting of bisphenol A (2,2-bis(4-hydroxyphenyl)propane), bisphenol AP (1,1-bis(4-hydroxyphenyl)-1-phenyl-ethane), bisphenol AF (2,2-bis(4-hydroxyphenyl)hexafluoropropane), bisphenol B (2,2-bis(4-hydroxyphenyl)butane), bisphenol BP (bis-(4-hydroxyphenyl)diphenylmethane), bisphenol C (bis(4-hydroxyphenyl)-2,2-dichloroethylene and also 2,2-bis(3-methyl-4-hydroxyphenyl)propane), bisphenol E (1,1-bis(4-hydroxyphenyl)ethane), bisphenol F (bis(4-hydroxyphenyl)methane), bisphenol G (2,2-bis(4-hydroxy-3-isopropylphenyl)propane), bisphenol M (4,4′-(1,3-phenylenediisopropylidene) bisphenol), bisphenol S (bis(4-hydroxyphenyl)sulfone), bisphenol P (4,4′-(1,4-phenylenediisopropylidene) bisphenol), bisphenol TMC (1,1-bis(4-hydroxyphenyl)-3,3,5-trimethylcyclohexane), bisphenol Z (1,1-bis(4-hydroxyphenyl)cyclohexane, and combinations thereof.

4. The benzoxazine oligomers of claim 1, wherein the aldehyde is formaldehyde added directly to or formed in situ in the reaction mixture.

5. The benzoxazine oligomers of claim 1, wherein the diamines or polyamine contain a compound selected from the group consisting of 4,4′-oxydianiline, 4,4′-diaminodiphenylmethane, 1,4-bis-(4-aminophenoxy)benzene, 4,4′-(p-biphenylenedioxy) dianiline, 4,4′-(9H-fluorene-9,9-diyl)dianiline, 2,7-bis(4-aminophenoxy)naphthalene, 1,3-bis(3-aminophenoxy)benzene, 1,3-bis(4-aminophenoxy)benzene, 1,4-bis(3-aminophenoxy)benzene, 1,3-bis(4-aminophenoxy)neopentane, 2,2-bis[4-(3-aminophenoxy)phenyl]propane, 2,2-bis[4-(4-aminophenoxy)phenyl]propane, 4,4′-bis(3-aminophenoxy)biphenyl, 4,4′-bis(4-aminophenoxy)biphenyl, 2,2-bis[4-(4-aminophenoxy)phenyl]hexafluoropropane, bis[3-(3-aminophenoxy)phenyl]ether, bis[3-(4-aminophenoxy)phenyl]ether, bis[4-(3-aminophenoxy)phenyl]ether, bis[4-(4-aminophenoxy)phenyl]ether, and combinations thereof.

6. The benzoxazine oligomers of claim 1 represented by the following formula: 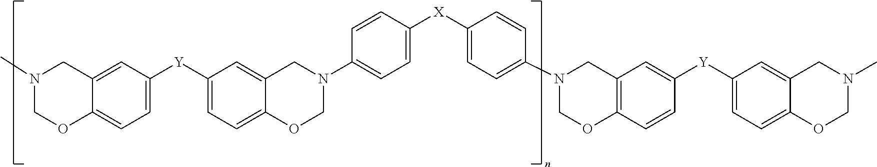
wherein n is a number from 1 to 15, X is oxygen, an alkyl, alkylene, alkylidene or arylene group, an alkylenedioxy group or a heteroatom containing group, Y is an alkyl, alkylene, alkylidene, or arylene group, which may be substituted or halogenated, a heteroatom containing group, or a combination or subset thereof.

7. The benzoxazine oligomers of claim 6 wherein the reaction product is produced from a reaction mixture comprising the following components:

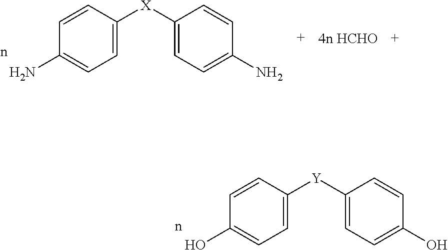

8. The benzoxazine oligomers of claim 6 wherein X is selected from one or more of the following groups:

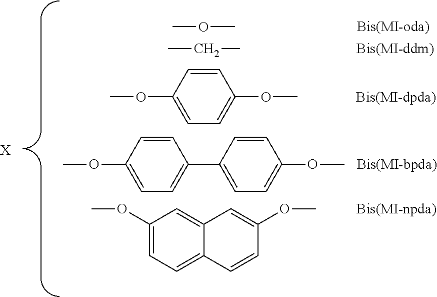

9. The benzoxazine oligomers of claim 6 wherein the reaction mixture comprises bisphenol-F, paraformaldehyde, and methylene dianiline.

10. The benzoxazine oligomers of claim 1 having a number average molecular weight (Mₙ) of between about 300 and about 10,000.

11. A reactive diluent comprising benzoxazine monomers, wherein the benzoxazine monomers comprise:
a reaction product of a bisphenol, a monofunctional amine, and an aldehyde present in a mole ratio of about 0.8:1.6:3.3 to about 1.2:2.4:4.8; wherein the monofunctional amine is a monoprimary aliphatic or aromatic amine containing between about 2 to about 50 carbon atoms and optionally contains one or more of ether, thioether, secondary amine, or tertiary amine linkages; or
a reaction product of a diamine, a monofunctional phenol, and an aldehyde present in a mole ratio of about 0.8:1.6:3.3 to about 1.2:2.4:4.8; wherein the monofunctional phenol comprises phenol or a substituted or unsubstituted phenolic compound.

12. A method of preparing main-chain benzoxazine oligomers comprising contacting a bisphenol, an aldehyde and a diamine or polyamine in a mole ratio of about 0.8:0.8:3.3 to about 1.2:1.2:4.8, at a temperature of between about 50° C. and about 100° C., for 1 to 10 hours.

13. The method of claim 12, further comprising contacting a monofunctional amine or a monofunctional phenol, in an amount calculated such that the main-chain benzoxazine oligomers contain a non-reactive end-group.

14. The method of claim 12, wherein the bisphenol contains a compound selected from the group consisting of bisphenol A (2,2-bis(4-hydroxyphenyl)propane), bisphenol AP (1,1-bis(4-hydroxyphenyl)-1-phenylethane), bisphenol AF (2,2-bis(4-hydroxyphenyl)hexafluoropropane), bisphenol B (2,2-bis(4-hydroxyphenyl)butane), bisphenol BP (bis-(4-hydroxyphenyl)diphenylmethane), bisphenol C (bis(4-hydroxyphenyl)-2,2-dichloroethylene and also 2,2-bis(3-methyl-4-hydroxyphenyl)propane), bisphenol E (1,1-bis(4-hydroxyphenyl)ethane), bisphenol F (bis(4-hydroxyphenyl)methane), bisphenol G (2,2-bis(4-hydroxy-3-isopropylphenyl)propane), bisphenol M (4,4′-(1,3-phenylenediisopropylidene) bisphenol), bisphenol S (bis(4-hydroxyphenyl)sulfone), bisphenol P (4,4′-(1,4-phenylenediisopropylidene) bisphenol), bisphenol TMC (1,1-bis(4-hydroxyphenyl)-3,3,5-trimethylcyclohexane), bisphenol Z (1,1-bis(4-hydroxyphenyl)-cyclohexane), and combinations thereof.

15. The method of claim 12, wherein the aldehyde is formaldehyde added directly or formed in situ.

16. The method of claim 12, wherein the diamines or polyamine contains a compound selected from the group consisting of 4,4′-oxydianiline, 4,4′-diaminodiphenyl methane, 1,4-bis-(4-aminophenoxy)benzene, 4,4′-(p-biphenylenedioxy) dianiline, 4,4′-(9H-fluorene-9,9-diyl)dianiline, 2,7-bis(4-aminophenoxy)naphthalene, 1,3-bis(3-aminophenoxy)benzene, 1,3-bis(4-aminophenoxy)benzene, 1,4-bis(3-aminophenoxy)benzene, 1,3-bis(4-aminophenoxy)neopentane, 2,2-bis[4-(3-aminophenoxy)phenyl]propane, 2,2-bis[4-(4-aminophenoxy)phenyl]propane, 4,4′-bis(3-aminophenoxy)biphenyl, 4,4′-bis(4-aminophenoxy)biphenyl, 2,2-bis[4-(4-aminophenoxy)phenyl]hexafluoropropane, bis[3-(3-aminophenoxy)phenyl]ether, bis[3-(4-aminophenoxy)phenyl]ether, bis[4-(3-aminophenoxy)phenyl]ether, bis[4-(4-aminophenoxy)phenyl]ether, and combinations thereof.

17. The method of claim 12 represented by: 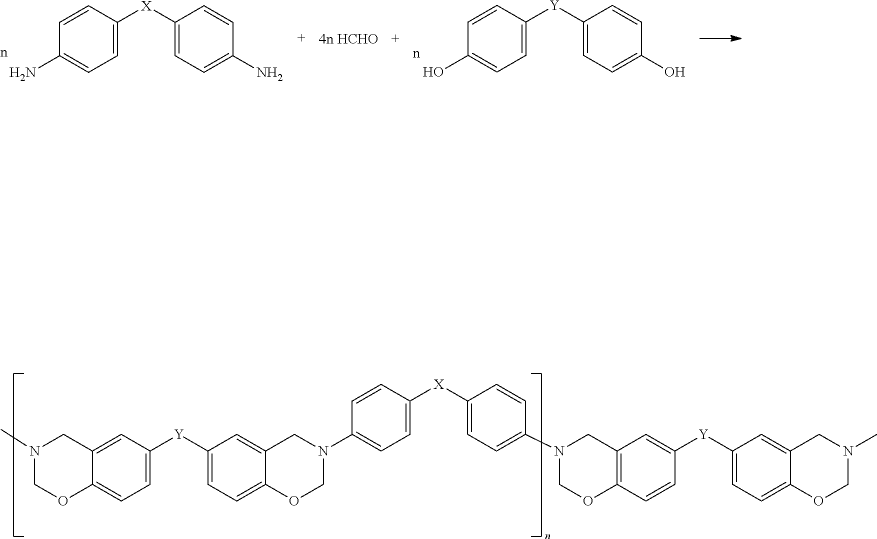
wherein n is a number from 1 to 15, X is oxygen, an alkyl, alkylene or alkylidene group, an alkylenedioxy group or a heteroatom containing group, Y is an alkyl, alkylene alkylidene, or aryl group, which may be substituted or halogenated, a heteroatom containing group, or a combination or subset thereof.

18. The method of claim 17 wherein X is selected from one or more of the following groups:

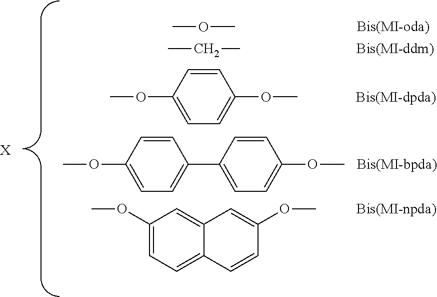

19. The method of claim 17 wherein the bisphenol comprises bisphenol-F, the aldehyde is paraformaldehyde, and the diamine or polyamine is methylene dianiline.

20. A resin system comprising the benzoxazine oligomers of claim 1.

21. A structural composite manufactured utilizing the resin system of claim 20.
